(19) United States
(12) Patent Application Publication
(10) Pub. No.: US 2002/0164672 A1
(43) Pub. Date: Nov. 7, 2002

(54) Regulation of JNK activity by modulation of the interaction between the endocytic protein endophilin and the germinal center kinase-like kinase

(76) Inventors: Peter S. McPherson, Lachine (CA); Antoine Rachid Ramjaun, Montreal (CA)

Correspondence Address: GOUDREAU GAGE DUBUC, 800 PLACE VICTORIA, SUITE 3400, MONTREAL, QUEBEC, H4Z 1E9, CA

(21) Appl. No.: 09/843,245

(22) Filed: Apr. 25, 2001

(51) Int. Cl. G01N 33/53; G01N 33/537; G01N 33/543; C12Q 1/48

(52) U.S. Cl. 435/15; 435/7.92

(57) ABSTRACT

Endophilin I is a brain-specific protein functioning in clathrin-mediated endocytosis. The present invention is based on the finding that the rat germinal center kinase-like kinase (rGLK), a member of the germinal center kinase (GCK) family of c-jun N-terminal kinase (JNK) activating enzymes, is a novel endophilin I-binding partner. In a first aspect of the present invention, the novel interaction between endophilin I and rGLK is put to use in a novel screening assay. In a second aspect of the present invention, the interaction between endophilin I and GLK is modulated for therapeutic purposes, namely for the prevention and/or curtailment of neurological disorders associated with the JNK pathway. JNK-mediated neuronal cell death is believed to play an important role in injuries and diseases involving neuronal degeneration, such as Huntington's disease.

### FIELD OF THE INVENTION

The present invention relates to the discovery of a novel interaction between endophilin I and the germinal center kinase-like kinase (GLK). In a first aspect of the present invention, the interaction between endophilin I and GLK is put to use in a novel screening assay. In a second aspect of the present invention, the interaction between endophilin I and GLK is modulated for therapeutic purposes, namely for the prevention and/or curtailment of neurological disorders associated with the JNK pathway. JNK-mediated neuronal cell death is believed to play an important role in injuries and diseases involving neuronal degeneration, such as Huntington's disease.

### BACKGROUND OF THE INVENTION

MAPK Intracellular Signaling Pathways: The JNK Pathway

Eukaryotic cells transmit extracellular stimuli into the cell through signaling pathways that employ cascades of specific protein kinases. One group of such signaling cascades have been collectively called the mitogen-activated protein kinase (MAPK) pathways. At present, there are at least six MAPK pathways identified in mammalian cells. The most studied is the extracellular signal regulated kinase (ERK) pathway which is activated by a host of stimuli including mitogenic factors. Other MAPK pathways are not necessarily activated in response to mitogens. For example, the c-Jun N-terminal kinase (JNK) pathway is activated in response to specific environmental stresses (e.g. osmotic, redox, radiation, and ischemia), and various inflammatory cytokines (e.g. tumor necrosis factor).

The JNK signaling pathway is involved in the control of a wide variety of physiological and pathological conditions including development, cell death, inflammation and response to ischemic injury. This involvement in a diverse array of cellular responses appears to depend upon the unique characteristics of the cell type in which JNK pathway activation is occurring, as well as on the ‘cross-talk’ with other MAPK signaling pathways. Unfortunately, because of these complex interactions with other MAPK pathways, it has been exceedingly difficult in most cell types to determine the exact impact that JNK activation has on a cell.

A major effect associated with the JNK pathway in cells in the nervous system is the activation of programmed cell death (apoptosis). JNK-mediated neuronal cell death is believed to play an important role in injuries and diseases involving neuronal degeneration.

GLK-endophilin Interactions

The individual elements of the JNK signaling pathway itself have been intensively studied, and many of the more downstream elements in the pathway have been delineated. However, the more upstream members and the mechanisms by which events at the cell surface are linked to activation of the downstream members have remained more elusive. One family of candidate protein kinases that appear to function as upstream members in the pathway are the recently described germinal center kinases (GCKs) of which GLK is a member.

The endophilin family, consisting of three highly homologous members referred to as endophilins I, II, and III, have been strongly linked to endocytosis. Over recent years, several studies have suggested a link between endocytosis and the regulation of MAPK signaling pathways This interaction serves to provide yet another link between the two cellular processes although it is amongst the first to link endocytosis to JNK signaling. It has been demonstrated both in vitro and in vivo that endophilin I interacts with GLK (Ramjaun et al., submitted, unpublished observations). All of the endophilins interact with GLK in vitro. Endophilin I (and most likely endophilins II and III) can regulate the JNK pathway (Ramjaun et al., submitted). Consequently, the GLK-endophilin interactions are likely to be physiological components of the GLK-mediated JNK pathway.

In addition, most GCKs are expressed in a wide variety of tissues, however, GLK is predominantly expressed in the brain. The protein appears to be expressed both in neurons and in glia. Within the brain, the neuronal expression is limited to specific neuronal populations. For example, the CA3 and CA2 pyramidal neurons in the hippocampus, the neurons of the substantia nigra pars compacta of the basal ganglia, the medial septum and the deep cerebellar nuclei all express high levels of GLK, whereas other regions of the brain, such as the CA1 neurons and the dentate granule cells of the hippocampus, or the substantia nigra pars reticulata, express little or no GLK (Ramjaun et al., unpublished observations). Intriguingly, many of the GLK-expressing neuronal populations are susceptible to JNK-mediated neuronal degeneration in various models of disease. For example, it has long been appreciated that the CA3 neurons of the hippocampus are prone to excitotoxic cell death (Nadler et al., 1980), a process mediated through activation of the JNK pathway (Yang et al., 1997), and which is a model for a number of neurological pathologies including Huntington's disease and ischemic brain injury. In addition, the substantia nigra pars compacta undergoes JNK-mediated apoptotic cell death following axonal damage (Herdegen et al., 1998; Oo et al., 1999) or treatment of nigral neurons in vitro with the dopaminergic neuron toxin MPTP (Saporito et al., 1999) both of which are models for Parkinson's disease.

An Additional Link: A Potential Role for GLK-endophilin Interactions in Huntington's Disease

A disease in which the link between endophilin and the JNK pathway may be important is in the neurological disorder known as Huntington's disease (HD). HD is caused by an expansion of a trinucleotide repeat (encoding polyglutamine) in the huntingtin protein of HD patients. Interestingly, it has been recently demonstrated that expression of this pathological form of huntingtin in a hippocampal neuronal cell line induces apoptosis via JNK activation (Liu, 1998; Liu et al., 2000), suggesting a role for JNK activation in the pathophysiology of HD. Interestingly, endophilin III has been recently identified to interact directly with huntingtin through a proline-rich sequence that is adjacent to its polyglutamine region. Endophilin III appears to interact with greatly increased affinity when huntingtin is expressed in its pathological form (Sittler et al., 1998). Since endophilin III can interact with GLK, it is intriguing to speculate that this may represent the mechanism through which huntingtin is able to regulate the JNK pathway. This may be through competition of the pathological form of huntingtin with GLK for binding to endophilin I and/or III, leading to a disregulation of JNK signaling via disruption of GLK-endophilin I/III interactions. In addition, an interesting feature of the huntingtin protein is that although it is broadly distributed in the nervous system and other tissues, only limited neuronal populations within the brain show an HD pathology. GLK and endophilin III demonstrate a restricted distribution in neurons including neuronal populations in the striatum, cortex and mid-brain that may correspond to populations particularly vulnerable to cell death in HD (unpublished observations; Sittler al., 1998).

Src Homology 3 (SH3) Domains:

Src homology 3 (SH3) domains are protein modules that bind to specific proline-rich sequences (Kay et al, 2000). SH3 domains are found in numerous proteins that control the subcellular targeting of proline-rich enzymes regulating signal transduction pathways (Kay et al., 2000), and SH3 domain mediated protein-protein interactions also function in vesicular trafficking, particularly in endocytosis (McPherson, 1999). Interestingly, recent studies have suggested a link between endocytosis and regulation of signal transduction pathways. For example, the ability of multiple tyrosine kinase and G-protein-coupled receptors to activate erk½ MAP kinase appears to require a clathrin-mediated endocytic step (reviewed in Ceresa and Schmid, 2000). The identification of intersectin and amphiphysin II, SH3 domain-containing proteins that function in endocytosis, as binding partners for the Ras activating enzyme MSos (Leprince et al., 1997; Tong et al., 2000), may suggest a mechanisms by which endocytosis and erk½ signaling are linked.

Another SH3 domain-containing protein that functions in clathrin-mediated endocytosis is endophilin I (de Heuvel et al., 1997; Micheva et al., 1997a; Ringstad et al., 1997). Endophilin I is a brain-specific member of a family of three highly related proteins that includes endophilin II, which is ubiquitously distributed, and endophilin III, which is expressed in brain and testis (Sparks et al., 1996; Giachino et al., 1997; Micheva et al. 1997a; Ringstad et al., 1997; So et al., 1997). Overexpression of the C-terminal SH3 domain of endophilin I in cell permeable assays (Simpson et al., 1999) or in lamprey synapses (Ringstad et al., 1999; Gad et al., 2000) blocks clathrin-coated vesicle formation.

Through its SH3 domain, endophilin I has been reported to interact with a variety of partners including the endocytic proteins synaptojanin 1, dynamin I, and the amphiphysins (Ringstad et al., 1997; Micheva et al., 1997a, 1997b), as well as the β1-adrenergic receptor (Tang et al., 1999) and specific metalloprotease disintegrins (Howard et al, 1999).

### SUMMARY OF THE INVENTION

Endophilin I is a brain-specific protein functioning in clathrin-mediated endocytosis. Here, we have identified and cloned the rat germinal center kinase-like kinase (rGLK), a member of the germinal center kinase (GCK) family of c-jun N-terminal kinase (JNK) activating enzymes, as a novel endophilin I-binding partner. The interaction occurs both in vitro and in vivo and is mediated by the SH3 domain of endophilin I and a region of rGLK containing the sequence PPRPPPPR. Unlike other members of the GCK family, rGLK is expressed predominantly in brain where it co-immunoprecipitates with endophilin I. In cultures from the CA3 region of the hippocampus, rGLK is detected in both neurons and glia. Within the neurons, rGLK is detected in puncta that extend into the neurites including the growth cone. In brain, expression of rGLK in neurons is restricted to specific neuronal populations, such as the CA3 pyramidal neurons, which are vulnerable to JNK-mediated neuronal cell death. Importantly, overexpression of full-length endophilin I activates rGLK-mediated JNK activation in HEK-293 cells, whereas N- and C-terminal fragments of endophilin I block JNK activation. Thus, endophilin I appears to have a novel function in JNK activation.

General Purpose and Commercial Applications

The GLK-endophilin interaction is a novel interaction that has not been described previously. It provides new understanding of the JNK signaling pathway, which in the nervous system is important in neuronal degeneration. This interaction may be important in an number of pathological conditions, and may provide a novel target for compounds to therapeutically treat these diseases.

More specifically, an example through which this intellectual property may be used in practice is as follows:

One of the proteins (for example, GLK) may be immobilized on a 96 well microtiter plate and incubated with a soluble form of the corresponding interacting protein (endophilin). This may then be used to screen drug libraries to determine candidate compounds that regulate the interaction and may therefore be used in therapeutic applications in either Huntington's disease or other neurological diseases/injuries involving JNK-mediated cell death. These candidate compounds could then be further screened using in vivo systems (similar to the ‘JNK assay.’ described in our article for submission) for their practical use in modulating the activation of the JNK pathway in specific neurons that may be undergoing stress-induced or disease-induced neuronal cell death.

Advantages and Improvements Over Existing Technologies

The major advantages that this interaction possesses over existing technologies, are as follows.

Firstly, this interaction has not been described previously, so it has not been previously accessible to manipulation.

Secondly, there appears to be convergence in the MAPK signaling pathways, so that when one moves further downstream, multiple extracellular stimuli activate the same set of more downstream elements. Thus, the more upstream members appear to be more pathway-specific. Consequently, since GLK is an upstream kinase, it allows for targeting of a very specific pathway.

Drug Screening Assay

The invention provides methods for detecting agents such as drugs that can alter the ability of members of the endophilin protein family to associate with the germinal center kinase-like kinase (GLK) protein, and methods for detecting agents that induce dissociation of a bound complex formed by the association of an endophilin family member and the GLK protein.

More specifically, in accordance with the present invention, there is provided a method for the identification of an agent that can alter the ability of an endophilin family member or endophilin fusion protein to associate with the germinal center kinase-like kinase (GLK) protein comprising the steps of:

(a) in a reaction mixture, allowing said endophilin family member or endophilin fusion protein, which is characterized by having an affinity for a solid substrate as well as having an affinity for GLK, to bind to a solid substrate;

(b) adding GLK together with an agent to be tested to the reaction mixture of (a) to form a second reaction mixture;

(c) allowing the second reaction mixture of (b) to incubate; and

(d) measuring the association of said endophilin family member or said endophilin fusion protein with GLK in the presence of said agent to be tested, and comparing same under conditions when said agent to be tested is absent from the second reaction mixture.

Other objects, advantages and features of the present invention will become more apparent upon reading of the following non restrictive description of preferred embodiments thereof, given by way of example only with reference to the accompanying drawings.

### OBJECTS OF THE INVENTION

An object of the present invention is therefore the application of the novel interaction between endophilin I and GLK in a novel screening assay.

A further object of the present invention is the modulation of the interaction between endophilin I and GLK for therapeutic purposes, namely for the prevention and/or curtailment of neurological disorders associated with the JNK pathway.

### BRIEF DESCRIPTION OF THE DRAWINGS

In the appended drawings:

FIG. 1 shows the sequence and structure of rGLK (A) The nucleic acid and complete coding sequence of rGLK is shown. The N-terminal sequence, determined from four overlapping rat EST clones is underlined. The bold amino acids represent consensus SH3 domain-binding sites. The nucleic acid numbers are indicated on the right. (B) rGLK is shown aligned to hGLK) and hGCK). The N-terminal kinase domain and C-terminal regulatory domain (including the proline-rich core) are indicated. The amino acid numbers of rGLK that define each domain are indicated along the top of the protein. The percent identity between the various domains is indicated.

FIG. 2 shows Endophilin I-rGLK interactions. (A) Triton X-100 soluble extracts were prepared from HEK-293T cells transfected with FLAG tagged rGLK or GCK (top panels) or with full-length endophilin I or endophilin I lacking the SH3 domain (endophilin I delta SH3) (bottom panels). The extracts were incubated with GST or with GST fusion proteins of either the SH3 domain of endophilin I (GST-SH3) or a portion of the proline-rich regulatory domain of rGLK (GST-GLK₂₇₆₋₅₄₁) pre-bound to glutathione-Sepharose. Proteins specifically bound to the beads (beads) were processed for Western blots along with an aliquot of the soluble extract (starting material, sm) and an equal amount of the unbound material (void). The panels show immunoblots with anti-FLAG (upper panels) or anti-endophilin I (bottom panels) (B) Triton X-100 soluble extracts of HFK-293T cells, co-transfected with FLAG-rGLK and either full-length endophilin I or endophilin I lacking the SH3 domain (endophilin I delta SH3), were used for immunoprecipitation analysis using anti-FLAG, anti-endophilin I (1903), or pre-immune 1903 (pre-immune). Proteins specifically bound to the beads were processed for Western blots with anti-FLAG (upper pannel) or anti-endophilin I (bottom pannel) antibodies. (C) A GST fusion protein encoding the proline-rich core of rGLK (GST-GLK₂₇₆₋₅₄₁), conjugated to glutathione-Sepharose beads, was incubated with soluble extracts from rat brain. Proteins specifically bound to the beads (beads) along with equal aliquots of the soluble brain extract (starting material, sm) and unbound material (void) were processed for Western blots with an antibody recognizing amphiphysin I and II (top panel) or endophilin I (bottom panel). For all blots, the migratory positions of the various brain proteins are indicated on the left.

FIG. 3 shows the identification of the endophilin I-binding site on rGLK. (A) Domain model of GST fusion protein constructs of the proline-rich core of rGLK. (B) Soluble extracts prepared from rat brain were incubated with the rGLK fusion proteins described in A. Proteins specifically bound to the beads (beads) along with aliquots of the soluble brain extract (starting material, sm), and equal amounts of the unbound material (void) were processed for Western blot with an antibody against endophilin I. The migratory position of endophilin I is indicated on the left. (C) Amino acid sequence alignment of a portion of the regulatory domains, including the proline-rich cores, of hGLK, rGLK, and hGCK. Homologous residues are lightly shaded, SH3 domain-consensus binding sites are more darkly shaded, and the likely endophilin I-binding site is darkly shaded.

FIG. 4 shows the rGLK overlay assays. Overlay assays using the GST-rGLK fusion protein constructs shown in FIG. 3A were performed on strips of rat brain post-nuclear supernatants immobilized on nitrocellulose. Coomassie Blue staining reveals the complement of proteins and an endophilin I antibody (1903) was used to indicate the migratory position of rat brain endophilin I. The migratory positions of molecular weight markers are indicated along the left.

FIG. 5 shows the characterization of an rGLK antibody. (A) An affinity-purified rabbit polyclonal antibody (2467), raised against GST-GLK₂₇₆₋₅₄₁, was used for Western blots of a crude extract of rat brain (brain) and on lysates of non-transfected HEK-293T cells (NT) or cells transfected with full-length, FLAG-tagged rGLK (FLAG-rGLK). A parallel transfer was blotted with anti-FLAG antibody. (B) A GST fusion protein encoding the SH3 domain of endophilin I (GST-SH3) or GST alone, conjugated to glutathione-Sepharose beads, were incubated with soluble extracts from rat brain. Proteins specifically bound to the beads (beads) were processed for Western blots with antibody 2467 along with equal aliquots of the soluble brain extract (starting material, sm) and unbound material (void). The migratory position of the 100 kDa rGLK band is indicated on the left.

FIG. 6 shows the endophilin I-rGLK interactions in brain. Post-nuclear supernatants from adult rat tissues and from embryonic day 18 rat brain (E18) were Western blotted with affinity purified antibodies against rGLK and endophilin I. In other experiments, soluble adult rat brain extracts were incubated with antibody 2467 or with pre-immune sera (NRS), pre-coupled to protein A-Sepharose. The proteins specifically bound to the beads were processed for Western blots with antibodies against rGLK and endophilin I. The migratory positions of the proteins are indicated on the left.

FIG. 7 shows the localization of rGLK in neurons. (A) rGLK is found in large puncta that are expressed throughout the cell body and neurites of hippocampal neurons from the CA3 region maintained in culture for two days. The puncta are concentrated in the peri-nuclear region and the proximal region of the dendrite (arrow head) and are also detected in the growth cone (arrow). (B) A higher magnification image of the growth cone in A reveals rGLK positive puncta (arrow). (C) Color-coding of fluorescent intensifies of the area in B indicates that the intensity of individual rGLK positive puncta is higher in the growth cone (red color) than in other regions of the dendrite. Scale bar: (A)=10 μM, (B,C)=4.0 μM.

FIG. 8 shows the localization of rGLK in adult rat brain. (A) rGLK is strongly expressed in the CA3 region of the hippocampus but is not detected in the dentate granule cells (DG). rGLK is observed in scattered neurons in the hillus (H). (B) rGLK staining is seen to extend throughout the hippocampal CA3 region to the CA2 region. In contrast, little rGLK staining is detected in CA1 neurons. The inset shows a high magnification image of rGLK staining in CA3 pyramidal neurons. (C) rGLK is strongly expressed in neurons of the substantia nigra pars compacta (PC) but is not detected in the substantia nigra pars reticulata (PR).

FIG. 9 shows that endophilin I regulates rGLK-mediated JNK activation. (A) HEK-293T cells were transfected with control plasmid, FLAG-JNK, FLAG-rGLK, full-length endophilin I, endophilin I lacking the SH3 domain, or GFP-SH3 domain of endophilin I, either alone or in various combinations as indicated. Fourty-eight hours following transfections, the cells were scrapped and processed for western blots with anti-FLAG, anti-endophilin, or anti-PO4-JNK antibodies as indicated. (B) HEK-293T cells were transfected with FLAG-JNK, FLAG-rGLK, GFP, or GFP-SH3 domain of endophilin I as indicated. Fourty-eight hours following transfections, the cells were scrapped and processed for Western blots with anti-FLAG, anti-endophilin, or anti-PO4-JNK antibodies as indicated. For all blots the migratory positions of the proteins are indicated on the left.

FIG. 10 is a diagrammatic representation of a screening assay for detecting agents such as drugs that can alter the ability of members of the endophilin protein family to associate with the germinal center kinase-like kinase (GLK) protein.

FIG. 11 reveals the reduced survival of glioma cells in the presence of GLK. Glioma cell lines (A) U343 and (8) U373 (p53 null) were plated in 96-well plates and then infected with nothing (0), control GFP adenovirus (gfp, white bars), or GLK adenovirus (GLK, black bars) at 50, 100, 250 or 500 MOI and analyzed for survival by MTT dye incorporation.

### DESCRIPTION OF THE PREFERRED EMBODIMENTS

Introduction

The present invention relates to the discovery of a novel SH3 domain-mediated protein-protein interaction between the endophilin protein family and the protein kinase, germinal center kinase-like kinase (GLK). The sites of interaction have been identified for both proteins. The ability of endophilin to regulate the activity of GLK has also been established. Moreover, an antibody has been raised against GLK and used to identify that GLK is expressed in specific neuronal populations in the brain. rGLK is also detected in glial cells in hippocampal cells or cortical cells in culture. This interaction is likely to be important in GLK-mediated JNK activation in cells of the nervous system and may be directly involved in their apoptotic pathways. Additionally, there is supplementary evidence for a role of GLK-endophilin interactions in JNK-mediated neuronal degeneration, specifically in Huntington's disease.

To better understand interactions mediated by endophilin I, we screened a rat brain expression library with the endophilin I SH3 domain. We identified synaptojanin 1 and dynamin I, and surprisingly, we also identified and cloned a rat homologue of the human germinal center kinase-like kinase (hGLK). hGLK was originally identified based on its homology to members of the germinal center kinase (GCK) family of protein kinases (Diener et al., 1997) and is a group I GCK (Kyriakis, 1999). Group I GCKs are mitogen-activated protein kinase (MAPK) kinase kinase kinases (MAP4Ks) that function upstream of c-Jun N-terminal kinase (JNK) in a variety of cell types and are activated in response to environmental stress, treatment with inflammatory mediators of the TNF family, and the vascular responses to ischemia (Kyriakis, 1999). Group I GCKs are composed of an N-terminal kinase domain and a C-terminal regulatory domain with multiple SH3 domain consensus-binding sites (Kyriakis, 1999). The mechanisms by which events at the cell surface are linked to activation of GLK and other GCKs, and the role of SH3 domain-mediated interactions in these processes remain poorly understood.

The identification of rat GLK (rGLK) as an endophilin I-binding partner suggests a role for endophilin I in the regulation of GLK function. To explore this, we first confirmed the rGLK-endophilin I interaction in multiple systems in vitro and used co-immunoprecipitation analysis from transfected cells and brain extracts to demonstrate the interaction in vivo. In fact, overlay assays suggest that endophilin I is the major SH3 domain-binding partner for rGLK in brain. Importantly, we have found that endophilin I functions directly in GLK-mediate JNK activation. UnliKe most GCK family members, rGLK is expressed almost exclusively in brain where it is detected in glial cells and in neuronal populations that are known to undergo JNK-mediated apoptotic cell death in different models of neuronal stress and disease. Taken together, these data implicate endophilin I in the activation of JNK and provide evidence of a novel link between the endocytic machinery and the JNK signaling pathway.

Materials and Methods

cDNA Expression Library Screen

An oligo(dT) primed λZAP II rat brain cDNA expression library, size selected for clones greater than 4 kb (Snutch et al., 1990; gift from Dr. Terry Snutch, University of British Columbia), was plated at 20,000 plaque forming units per 150 mm² plate. Protein expression was induced using isopropyl-β-D-thiogalactopyranoside-soaked nitrocellulose filters that were then screened by overlay assays (McPherson et al., 1994) using a GST fusion protein encoding the SH3 domain of endophilin I (Micheva et al., 1997a). Positive plaques were purified by two additional rounds of screening and the cDNAs were isolated and identified by DNA sequence analysis. The longest clone encoding rGLK was completely sequenced on both strands.

Antibodies

Polyclonal antibodies against amphiphysin I/II (Ramjaun et al., 1997) and endophilin I (1903) (Micheva et al., 1997a) were previously described. Monoclonal anti-FLAG M2 and anti-phospho-JNK (Thr183/Tyr185) antibodies were purchased from Sigma and New England Biolabs, respectively. A polyclonal anti-rGLK antiserum was raised in rabbits against a GST fusion protein encoding amino acids 276 to 541 of rGLK (GST-GLK₂₇₆₋₅₄₁), and anti-rGLK antibodies were affinity-purified from the serum as described (Sharp et al., 1993) using a His6-tagged version of the fusion protein.

Generation of GST Fusion Protein Constructs

A GST fusion protein encoding the SH3 domain of endophilin I was previously described (Micheva et al., 1997a) GST fusion protein constructs of GLK, encoding various regions of the regulatory domain, were generated using an rGLK cDNA clone as a template in PCR reactions.

The reactions were performed with the forward primer 5′-GCGGGATCCCCTCTGACGAGGTCTTTG (nucleotides 826-843) and the following reverse primers:

| | |
|---|---|
| -GST-GLK₂₇₆₋₅₄₁, | 5′-GCGCCCGGGTCAGGCACAGTGGATTTT |
| CAAG (nucleotides 1605-1623); | |
| -GST-GLK₂₇₆₋₄₉₈, | 5′-GCGCCCGGGTCAGTTCGTGCCTCTCT |
| GCTC (nucleotides 1477-1494); | |
| -GST-GLK₂₇₆₋₄₄₅, | 5′-GCGCCCGGGTCACCCTGATGAGGGA |
| CATC (nucleotides 1319-1335); | |
| -GST-GLK₂₇₆₋₄₀₆, | 5′-GCGCCCGGGTCACAAGGTTGAATGTTTA |
| GAGTC (nucleotides 1198-1218). | |

The resulting PCR products were subcloned into the corresponding BamHI and SmaI sites of pGEX-2T (Pharmacia Biotech Inc.) and the resulting GST fusion proteins were expressed and purified using standard procedures.

Generation of Constructs for Mammalian Expression

Mammalian expression constructs for full-length endophilin I and endophilin I lacking the SH3 domain (delta SH3) were generated by digesting the corresponding GST fusion protein constructs (Micheva et al., 1997a) with BamHI and EcoRI, with the resulting inserts subcloned into the corresponding sites of pcDNA3 (Invitrogen). An N-terminal green fluorescent protein (GFP) tagged mammalian expression construct for the SH3 domain of endophilin I was generated by PCR using the full length cDNA (Sparks et al., 1996) as a template, with the forward primer 5′-GCGAGATCTCTCAGCCAAGAAGGGAATATC, and the reverse primer 5′-GCGGAATTCTCAATGGGGCAGAGCAACCAG. The resulting PCR product was digested with Bgl II and EcoRI, and subcloned into the corresponding sites of pEGFP-C2 (Clontech). To generate a construct encoding rGLK with an N-terminal FLAG-tag, PCR was performed with the forward primer 5′-GCGAAGCTTGCCACCATGGACTACAAAGACGATGACGACAAACC GCAGGAGGACTTCG (nucleotides 34-49), which includes an initiation ATG codon within the context of a Kozak consensus sequence (Kozak, 1991) and a FLAG epitope (DYKDDDDK), and the reverse primer 5′-GCGGGATCCTCACTGTGTGACAAAGGGATG (nucleotides 808-825) The resulting PCR product was digested with HindIII and KpnI and subcloned into the same sites at the 5′-end of the longest rGLK library clone in pBluescript. The resulting FLAG-tagged rGLK construct was then digested with HindIII and BamHI sites and the insert was subcloned into the same sites in pCDNA3. FLAG-tagged GCK (Yuasa et al., 1998) and JNK constructs were generous gifts from Dr. John Kyriakis (Massachusetts General Hospital), and from Dr. Nathalie Lamarche-Vane (McGill University), respectively.

Tissue Extracts and Binding Assays

Post-nuclear supernatants (PNS) of various adult rat tissues were prepared in buffer A (20 mM HEPES-OH, pH 7.4, 0.83 mM benzamidine, 0.23 mM phenylmethylsulfonylfluoride, 0.5 μg/ml aprotinin, and 0.5 μg/ml leupeptin) as described (Ramjaun et al., 1997). For GST fusion protein binding assays, PNS from adult rat brain was centrifuged at 45,000 rpm in a Sorval T-865 rotor for 1 hour at 4° C. and the soluble supernatant was diluted to 2 mg/ml in buffer A. Triton X-100 was added to 1% final and 1 ml aliquots were incubated o/n at 4° C. with approximately 25 μg of GST fusion proteins pre-bound to glutathione-Sepharose. The beads were subsequently washed three times in 1 ml of buffer A with 1% Triton X-100. eluted with SDS-PAGE sample buffer, and prepared for Western blot analysis. For binding assays from cultured cells, 10 cm² dishes of transfected HEK-293T cells were washed in phosphate-buffered saline (PBS) (20 mM NaH₂PO₄, 0.9% NaCl, pH 7.4) and scraped into buffer B (buffer A with 150 mM NaCl). The cells were sonicated and passed through a 25⅝ gauge needle, Triton X-100 was added to a final concentration of 1%, and following incubation for 20 minutes at 4° C., the samples were centrifuged at 75,000 rpm in a Beckman TLA 100.1 rotor to remove the insoluble material. Aliquots of the soluble supernatant were diluted to 2 mg/ml in buffer B with 1% Triton X-100 and 1 ml samples were incubated o/n at 4° C. with approximately 25 μg of GST fusion proteins pre-bound to glutathione-Sepharose. The bead samples were subsequently washed in buffer B with 1% Triton X-100 and prepared for SDS-PAGE as described above. Overlay assays were performed as described (McPherson et al., 1994).

Immunoprecipitation Assays

Extracts from HEK-293T cells, co-transfected with FLAG-rGLK and either endophilin I full length or endophilin I delta SH3, were prepared as described above and were pre-cleared with either protein A-Sepharose or protein G-agarose beads. The pre-cleared supernatants were then incubated with pre-immune serum or anti-endophilin I antibody (1903) coupled to protein A-Sepharose beads, or with anti-FLAG antibody coupled to protein G-agarose beads. After 5 hr at 4° C., the beads were washed extensively in buffer B with 1% Triton X-100 and prepared for Western blotting analysis. For immunoprecipitations from tissue, rat brains were homogenized in buffer A containing 0.3 M sucrose using a glass-Teflon homogenizer with 9 strokes at 900 rpm. The homogenate was centrifuged at 800×gₘₐₓ for 5 minutes and the supernatant was then centrifuged at 16,000×gₘₐₓ for 15 minutes. NaCl was added to 150 mM and the sample was incubated for 15 minutes at 4° C. before being centrifuged at 45,000 rpm in a Sorval T-865 rotor for 1 hour at 4° C. The resulting soluble material was made to 1% in Triton X-100 and was pre-cleared by incubation with protein A-Sepharose. The pre-cleared supernatants were then incubated with pre-immune serum or anti-rGLK antibody (2467) coupled to protein A-Sepharose beads. After 5 hr at 4° C., the beads were washed extensively in buffer B with 1% Triton X-100 and prepared for Western blotting analysis.

Immunofluorescence Analysis of Frozen Brain Sections and Hippocampal Neurons in Culture

Rats were anesthetized and perfused through the ascending aorta with 200 ml of 0.1M sodium phosphate monobasic, pH 7.4 (phosphate buffer; PB) followed by 400 ml of 3.5% paraformaldehyde in PB and 200 ml of 10% sucrose in PB. The brains were dissected out, cryoprotected in 30% sucrose, and 30 μm sections were prepared on a freezing microtome. The sections were then washed in 0.1M Tris, pH 7.4 (Tris buffer), permeabilized in 0.3% Triton X-100 for 10 minutes, blocked in 5% BSA and 5% NGS for 30 minutes, and incubated with primary antibody in Tris buffer containing 1% BSA for 2 to 3 days at 4° C. The sections were then washed in 0.1M Trs buffer with 1% BSA and 0.1% Triton X-100, incubated with fluorescent secondary antibody at room temperature for 2 hours in the same buffer, washed, mounted, and dehydrated before observation. Dissociated cell cultures were prepared from the CA3 region of hippocampi from P1 rat pups as described (Hussain et al., 1999).

JNK Assays

HEK 293T cells were co-transfected with FLAG-tagged JNK and a variety of constructs as indicated in the figure legends. Fourty-eight hours post transfection, the media was removed and the cells were scraped and boiled in sample buffer. The samples were separated on SDS-PAGE and used for western blot analysis

Results

Identification and Cloning of Rat Germinal Center Kinase-like Kinase as an Endophilin I-binding Protein

A key requirement in understanding the complete range of endophilin I functions is the identification of its full complement of SH3 domain-binding partners. We thus screened a rat brain expression library with a GST fusion protein encoding the SH3 domain of endophilin I. From a total of approximately six-hundred thousand clones screened, eighteen encoded potential endophilin I-binding proteins (data not shown). As expected, the majority of the clones (eight) encoded for synaptojanin 1. Only one clone was found to correspond to dynamin I. Interestingly, three independent, non-amplified isolates were found to encode for a rat homologue of the human serine/threonine protein kinase, germinal center kinase-like kinase (hGLK) (Diener et al., 1997)

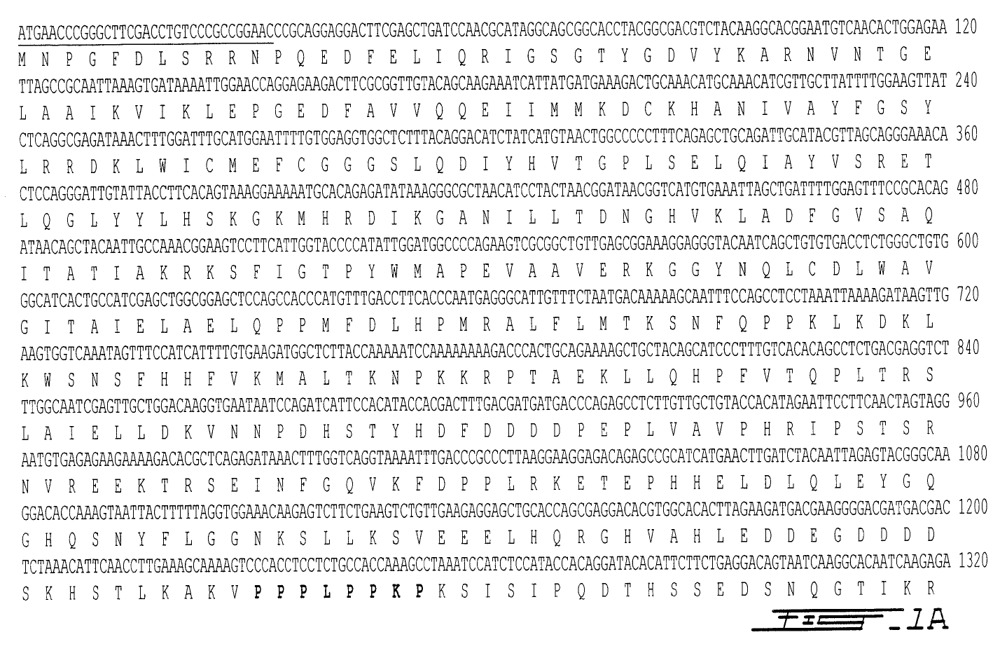

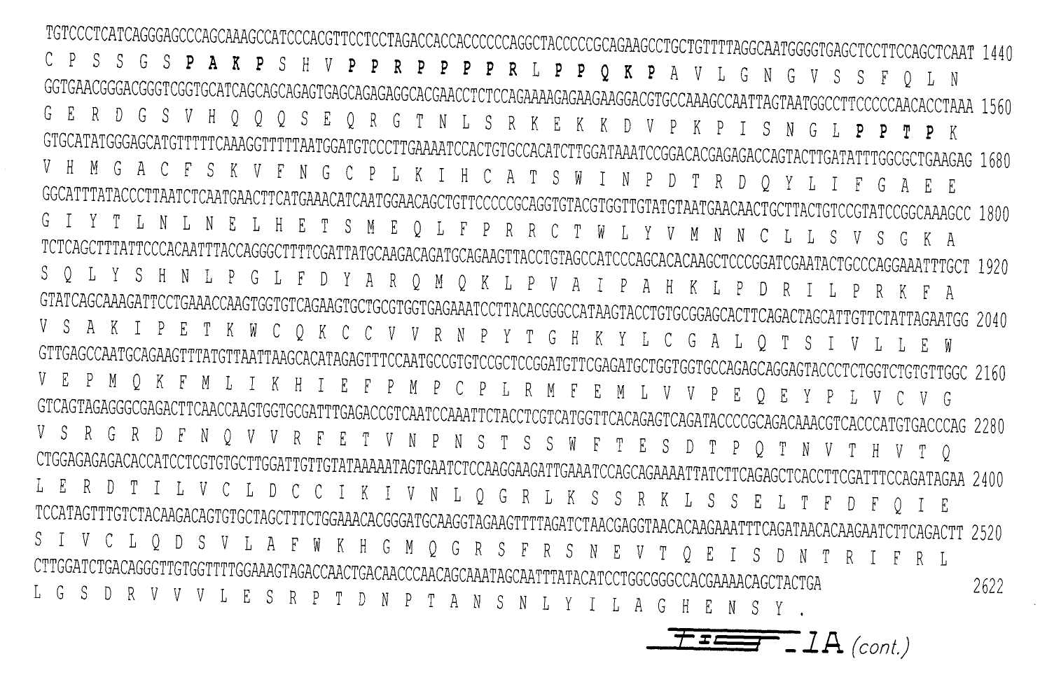

The longest rat GLK (rGLK) clone isolated was sequenced on both strands to generate a coding sequence that aligned to hGLK, starting at amino acid 3 of its published sequence (Diener et al, 1997), and appeared to be complete at the C-terminal end. To obtain the extreme 5′-end, we searched the database of expressed-sequence tags (dbEST) with sequence from near the 5′-end of our isolated clones. Four overlapping rat ESTs were identified leading to a complete rGLK coding sequence containing eleven additional amino acids (FIG. 1A). These amino acids, which extend the rGLK sequence eight amino acids beyond the predicted start of hGLK, were homologous to other members of the GCK family (data not shown). The differences in the extreme N-terminal end of hGLK and rGLK may represent a species difference. However, multiple human EST clones were identified, which when aligned with the 5′-end of hGLK, could define an N-terminal sequence identical to that for rGLK (data not shown).

Protein alignments revealed that the N-terminal kinase domain of rGLK is 99% and 72% identical to hGLK and hGCK, respectively (FIG. 1B). The C-terminal regulatory domain of rGLK, which includes a proline-rich region with multiple SH3 domain consensus-binding sites is 95% identical with hGLK and 44% identical with hGCK (FIG. 1B).

Endophilin I Interacts Specifically with rGLK through an SH3 Domain-mediated Interaction

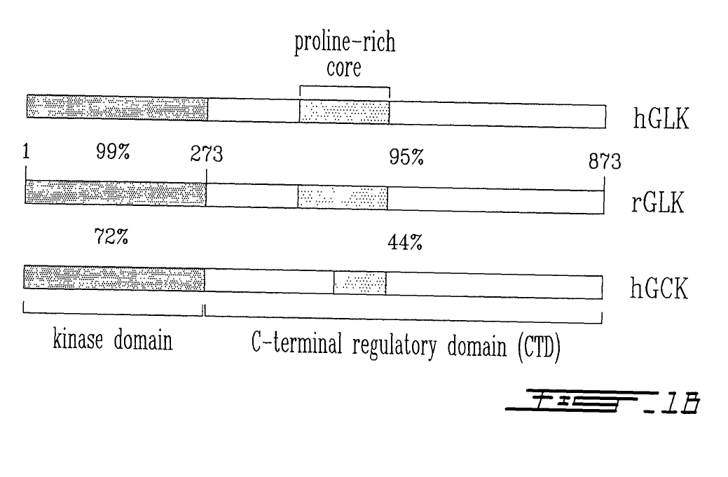

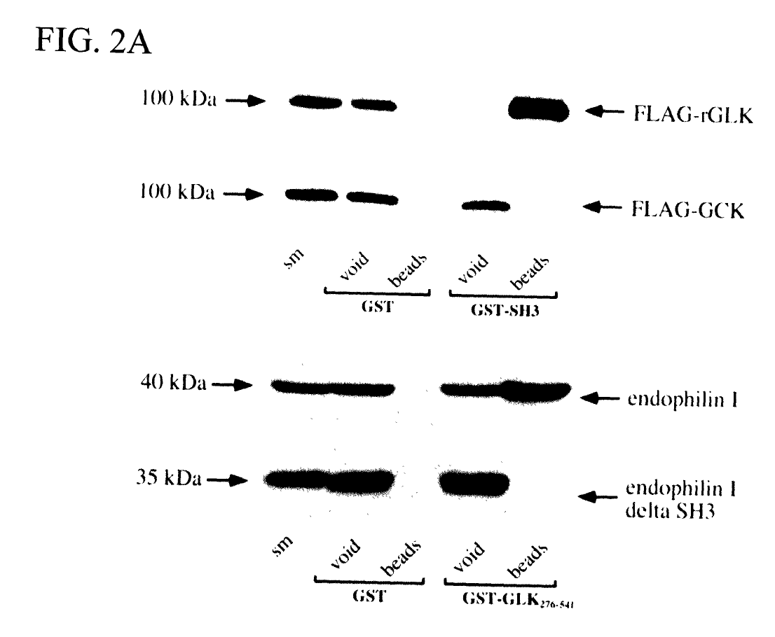

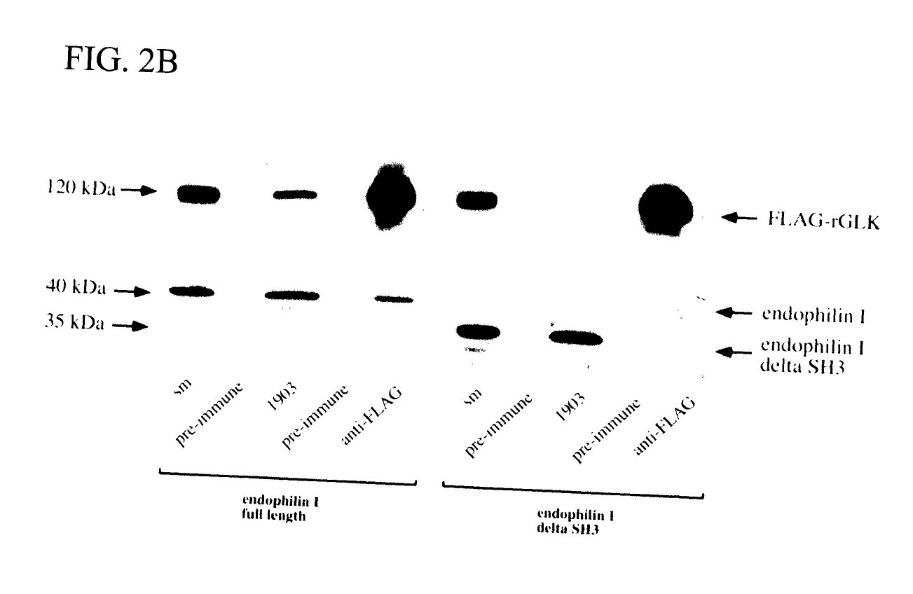

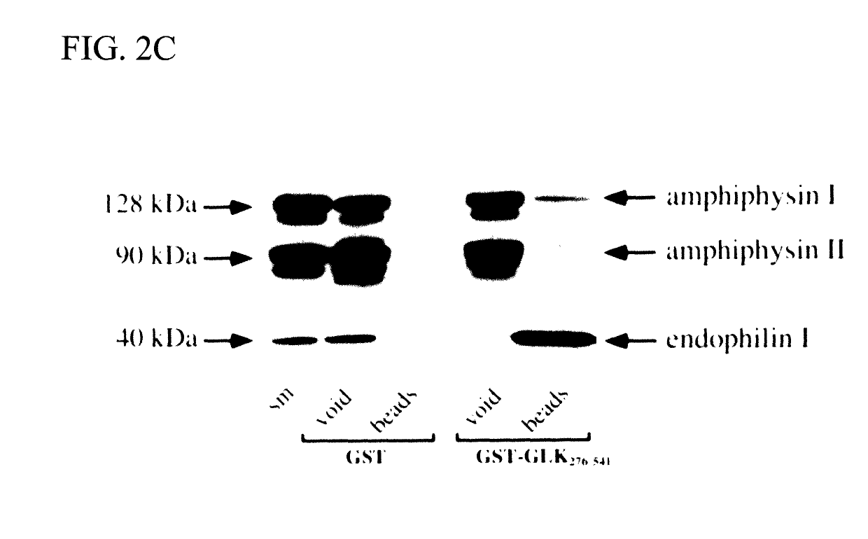

To confirm the endophilin I-rGLK interaction, we transfected HEK-293T cells with a construct encoding rGLK containing an N-terminal FLAG tag (FLAG-rGLK). The expressed protein was shown to bind strongly to a GST-endophilin I SH3 domain fusion protein (GST-SH3) but not to GST alone (FIG. 2A, top panels). To further demonstrate the interaction specificity, we assessed the binding of FLAG-tagged GCK (Yuasa et al., 1998) (generously provided by Dr. John Kyriakis). No binding of FLAG-GCK to the SH3 domain of endophilin I was detected (FIG. 2A, top panels). We next performed the converse pull-down experiments. Full-length endophilin I, expressed in HEK-293T cells, binds specifically to a GST fusion protein encoding the proline-rich core of rGLK (GST-GLK₂₇₆₋₅₄₁; FIG. 2A, bottom panels). In contrast, an endophilin I construct lacking the SH3 domain (endophilin I delta SH3) did not bind to the same rGLK fusion protein (FIG. 2A, bottom panels).

To demonstrate the interaction in cells, lysates from HEK-293T cells, co-transfected with FLAG-rGLK and full-length endophilin I, were subjected to immunoprecipitation analysis with an anti-endophilin I antibody (1903) and an anti-FLAG antibody. Immunoprecipitation of either endophilin I or FLAG-rGLK led to the co-immunoprecipitation of the respective binding partner (FIG. 2B). In control experiments, no co-immunoprecipitation was seen when cells were co-transfected with FLAG-rGLK and an endophilin I construct lacking the SH3 domain (endophilin I delta SH3) (FIG. 2B).

The endophilin I-rGLK interaction was also demonstrated from brain tissue. The GST-GLK276-541 fusion protein strongly affinity-selected endophilin I from brain extracts, whereas amphiphysin I and II, two major SH3 domain-containing proteins in brain (Ramjaun et al., 1997), did not interact with the fusion protein (FIG. 2C).

Identification of the Endophilin I-binding Site in rGLK

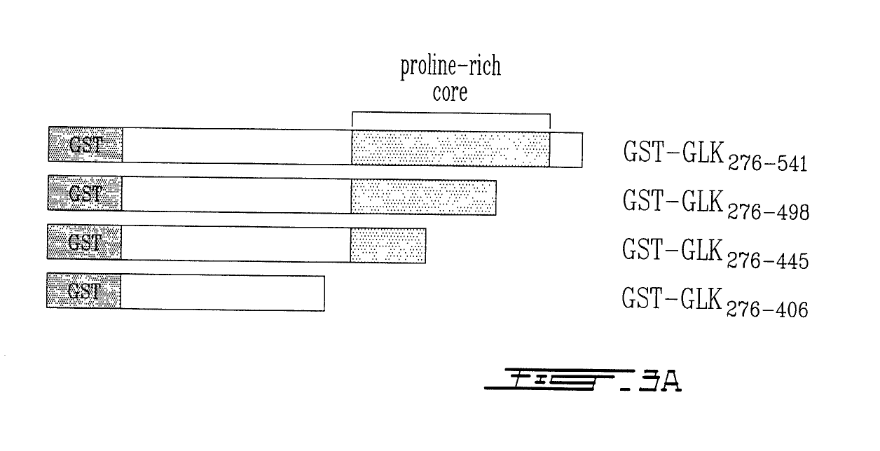

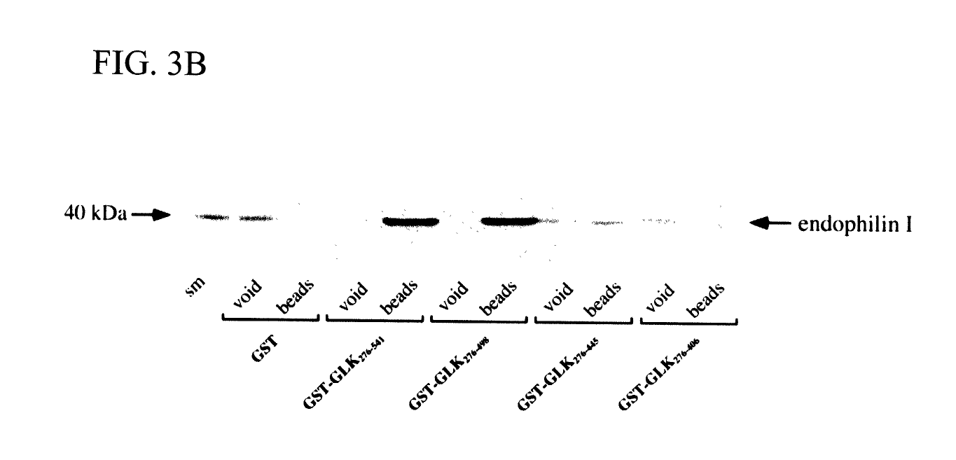

The proline-rich core of rGLK contains multiple consensus SH3 domain-binding sites (FIG. 3C). To identify the proline-rich motif(s) responsible for endophilin I binding, we generated GST fusion protein constructs in which we deleted increasing amounts of the C-terminus of the rGLK regulatory domain (FIG. 3A). Whereas a construct consisting of amino acids 276 to 498 of rGLK (GST-GLK₂₇₆₋₄₉₈) bound strongly to endophilin I from brain extracts, a construct encoding amino acids 276 to 445 (GST-GLK₂₇₆₋₄₄₅) showed weak binding and a construct encoding amino acids 276 to 406 (GST-GLK₂₇₆₋₄₀₆) failed to bind (FIG. 3B). This indicated that the major endophilin I-binding site was located between amino acids 445 and 498 of rGLK with a second, much weaker site between amino acids 405 and 446. Within the region of rGLK from amino acids 445 to 498 is the sequence PPRPPPPR (FIG. 3C), which closely conforms to the previously described SH3 domain-binding sequence preference for the endophilins (Cestra et al., 1999), suggesting that it is the major endophilin I-binding site in rGLK.

Endophilin I is a Major rGLK-binding Protein in Brain

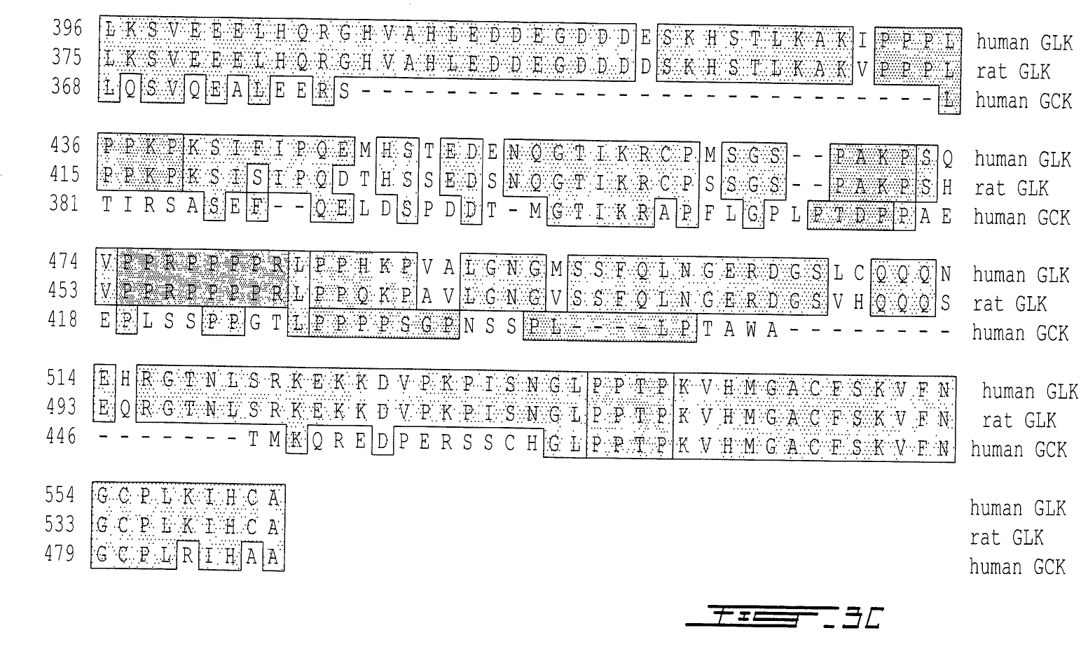

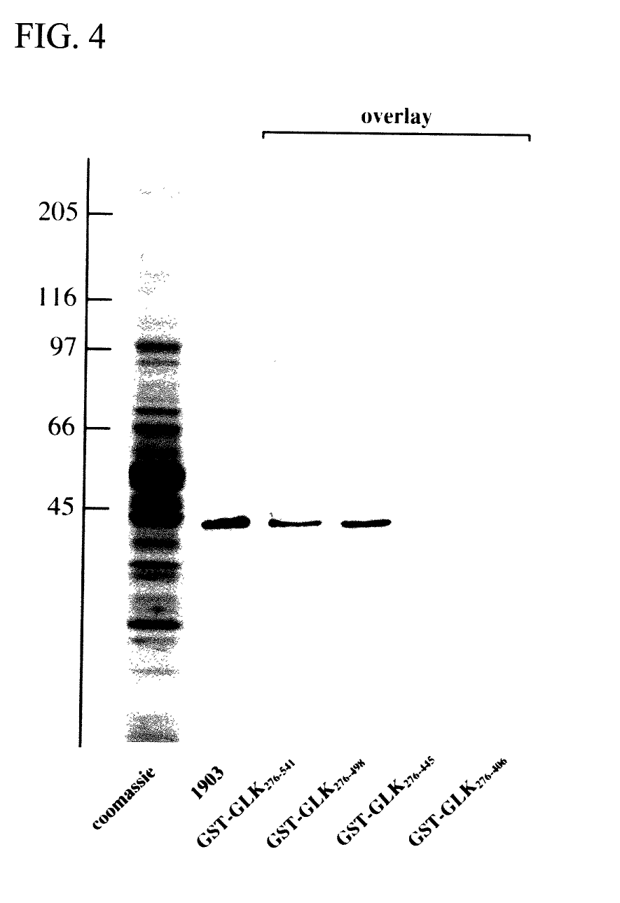

Members of the GCK family have been reported to interact with a number of different SH3 domain-containing proteins (Kyriakis, 1999). To determine whether rGLK has multiple SH3 domain binding partners, we performed overlay assays of brain extracts with the GST-rGLK fusion protein constructs described above. Remarkably, GST-GLK₂₇₆₋₅₄₁ reacted with a single 40 kDa band that perfectly co-migrated with endophilin I as determined by Western blot with the endophilin I antibody 1903 (FIG. 4). GST-GLK₂₇₆₋₄₉₈, which like GST-GLK₂₇₆₋₅₄₁ contains the major endophilin I-binding site, also bound to the 40 kDa band, whereas the fusion proteins lacking the endophilin I-binding sequence (GST-GLK₂₇₆₋₄₄₅, and GST-GLK₂₇₆₋₄₀₆) did not (FIG. 4). A similar specificity was seen with overlay assays of recombinant endophilin I (data not shown). Together, these data suggest that endophilin I is a major rGLK-binding protein in brain and provide compelling evidence for the specific nature of the endophilin I-rGLK interaction.

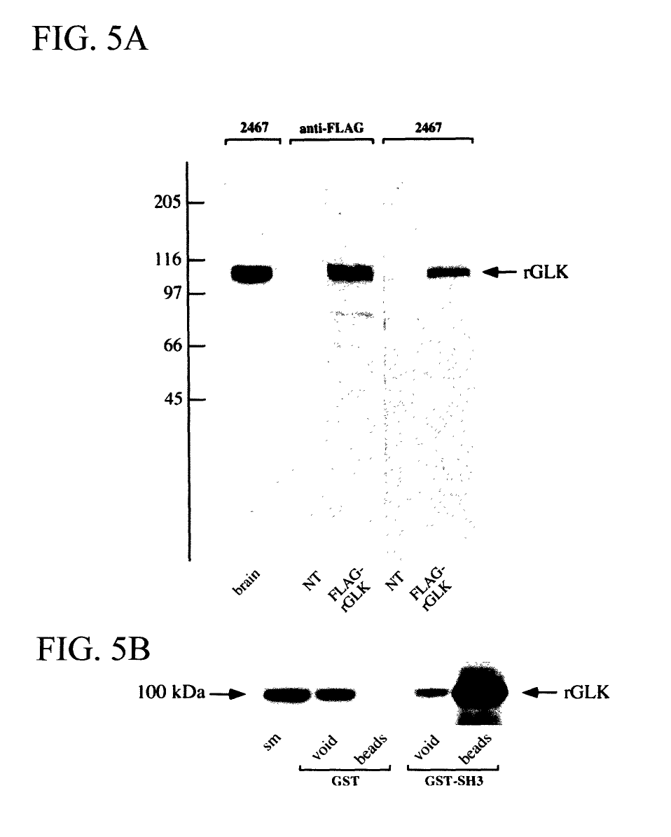

An anti-rGLK antibody was generated through injection of the GST-GLK₂₇₆₋₅₄₁ fusion protein. Following affinity purification, the antibody (2467) specifically recognized a 100 kDa band in rat brain extracts (FIG. 5A), consistent with the predicted molecular mass of 98,689 Da for rGLK. To confirm that the antibody recognizes rGLK, we transfected cells with FLAG-rGLK and blotted the cell lysates with anti-FLAG or anti-rGLK antibody 2467. Both antibodies recognized an identically sized band of ˜100 kDa in lysates from transfected cells but not in lysates from non-transfected cells (FIG. 5A). To further ensure that the 100 kDa band in brain extracts is indeed rGLK, we used the SH3 domain of endophilin I in affinity chromatography experiments. GST-SH3 but not GST alone strongly interacts with the 100 kDa band recognized by antibody 2467 (FIG. 5B).

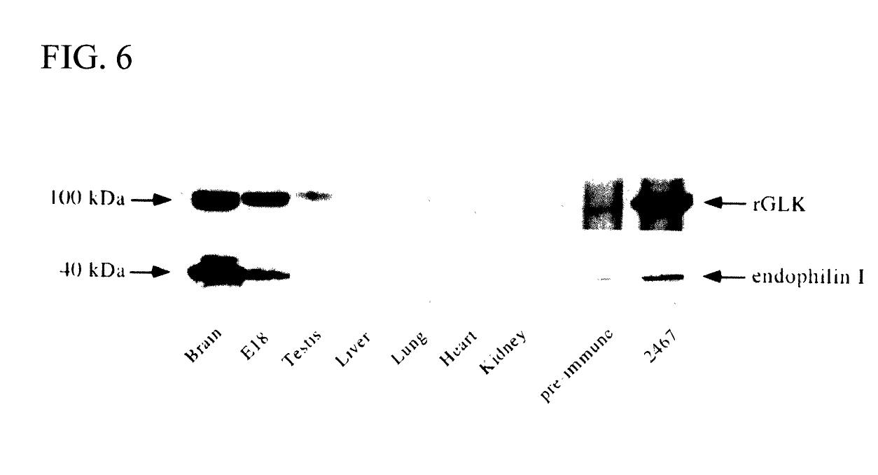

We next explored the tissue distribution of rGLK using the affinity-purified antibody. Previously reported Northern blot data suggested a ubiquitous distribution for the hGLK mRNA (Diener et al., 1997). Surprisingly, rGLK protein, like endophilin I (Micheva et al., 1997a), is strongly expressed in adult and embryonic day 18 (E18) brain and is weakly expressed in testis (FIG. 6), but is only seen in a variety of non-neuronal tissues upon extensive over-exposure of the blots (data not shown). To demonstrate that endophilin I interacts with rGLK in brain tissue, we immunoprecipitated rGLK from solubilized rat brain extracts using anti-sera 2467 and blotted the resulting immunoprecipitates with rGLK and endophilin I antibodies. Anti-sera 2467 immunoprecipitates rGLK and leads to co-immunoprecipitation of endophilin I (FIG. 6).

Immunofluorescence Analysis of the Distribution of rGLK in Neurons

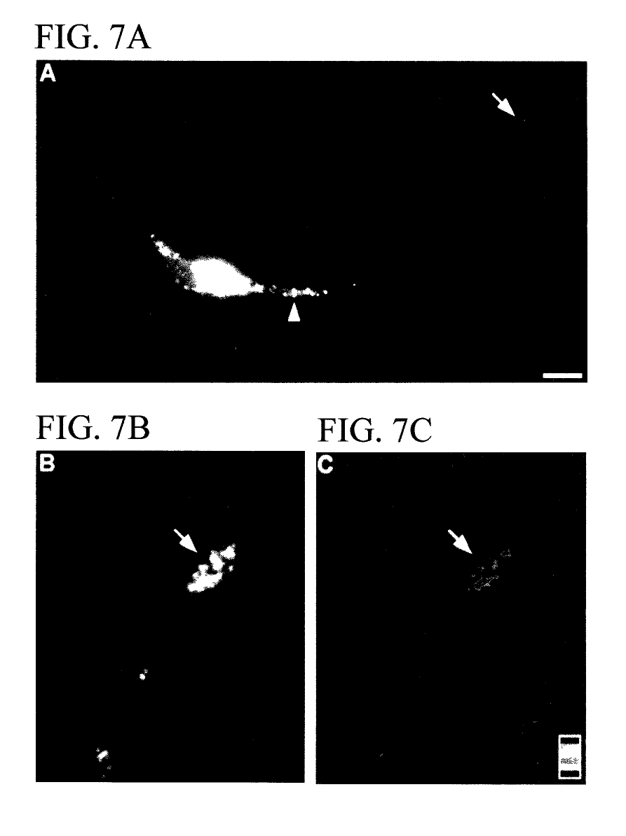

Western blot analysis of dissected brain regions revealed strong expression of rGLK in the hippocampus (data not shown). Thus, to determine if rGLK was expressed in neurons, we performed immunofluorescence analysis on neuronal cultures prepared from the CA3 region of the hippocampus. Interestingly, rGLK was detected in neurons, predominantly in large, punctate structures, possibly representing large vesicular elements (FIG. 7A). The puncta were concentrated in the peri-nuclear region but they also extended into dendrites and their growth cones (FIG. 7B). The enrichment in growth cones, which was only detected in a fraction of the neurons, was due to an increase in the density of rGLK positive puncta in the growth cone area (FIG. 7B) as well as to an increase in the content of rGLK per puncta (FIG. 7C).

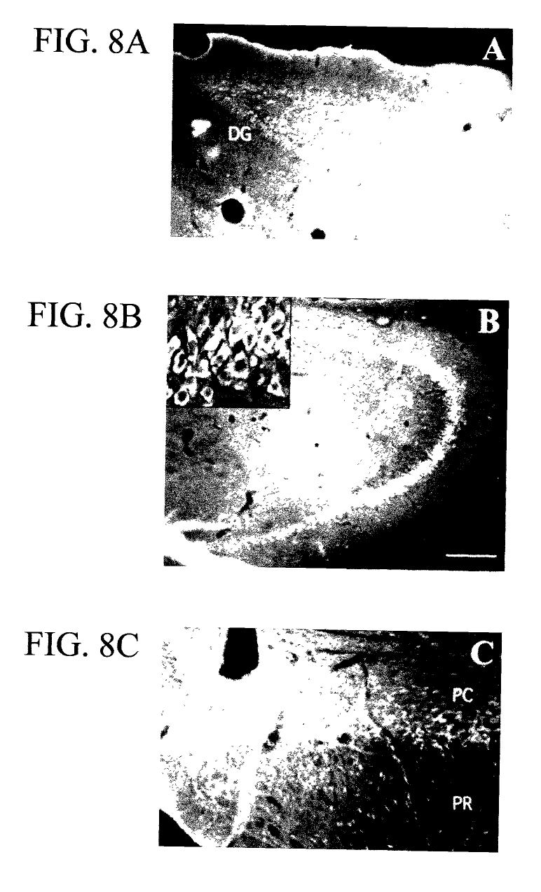

We next examined the regional distribution of rGLK in adult rat brain. rGLK was detected in specific neuronal populations in the cerebral cortex, the cerebellum, and the mid brain (data not shown). In the hippocampus, rGLK was strongly expressed in the CA3 and CA2 pyramidal neurons (FIGS. 8A,B) and in neurons from the hillus (FIG. 8A), but was only weakly detectable in CA1 pyramidal cells (FIG. 8B) and was not detected in dentate granule cells (FIG. 8A). rGLK was strongly expressed in the substantia nigra pars compacta but was absent from the substantia nigra pars reticulata (FIG. 8C). Higher power images of CA3 pyramidal neurons reveals staining that is present throughout the cell body and proximal dendrites (FIG. 8B inset).

Endophilin I Regulates GLK-mediated JNK Activation

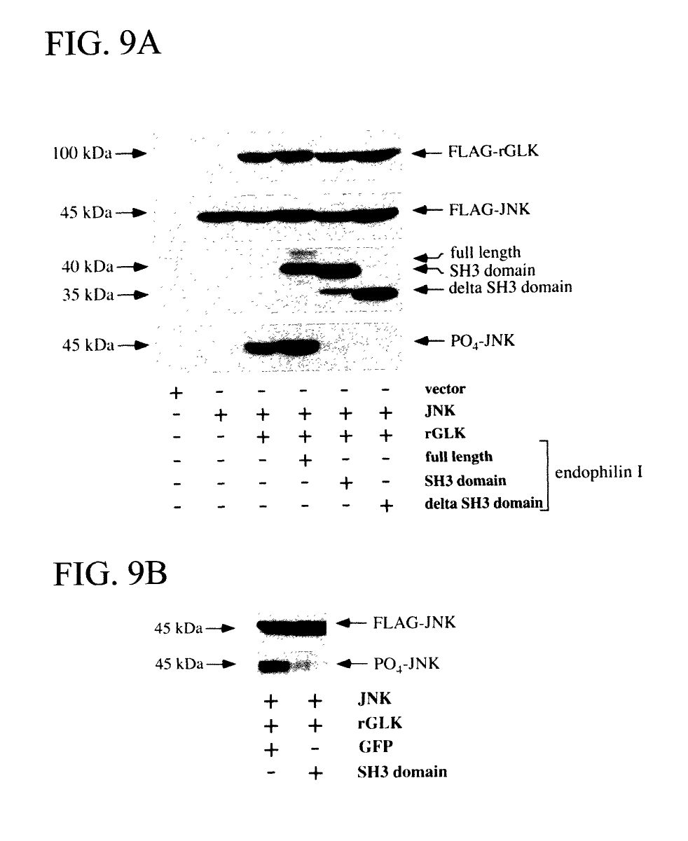

It was previously reported that overexpression of hGLK leads to JNK activation in transfected HEK-293 cells (Diener et al., 1997). We thus used this system to determine if endophilin I functions in GLK-mediated JNK activation. Specifically, we monitored JNK activation using an anti-phospho JNK antibody following transfection of GLK and different endophilin I constructs. Consistent with Diener et al., (1997), we find that overexpression of rGLK is sufficient to activate JNK (FIG. 9A). Interestingly, overexpression of full-length, untagged endophilin I along with rGLK leads to an increase in JNK activation versus expression of GLK alone (FIG. 9A). Quantitative analysis of 8 independent experiments revealed a statistically significant (p<0.05; two-tailed t-test) 2.01-fold stimulation. Further, overexpression of a GFP-tagged form of the SH3 domain of endophilin I or of the untagged N-terminus of endophilin I lacking the SH3 domain (delta SH3), completely blocked GLK-mediated JNK activation (FIG. 9A). Given that the SH3 domain of endophilin I was expressed with a GFP tag, we sought to further demonstrate the specificity of this construct to block rGLK-mediated JNK activation. Thus, JNK activation was measured following overexpression of rGLK with the GFP-SH3 domain construct or with GFP alone. Whereas the GFP-endophilin I SH3 domain blocked rGLK-mediated JNK activation, GFP alone had no effect (FIG. 9B).

Discussion

Endophilin I is a brain-specific protein implicated in clathrin-mediated endocytosis, in part through its SH3 domain-dependent interactions with synaptojanin 1, dynamin I, and amphiphysin I and II. In order to identify additional binding partners for endophilin I, we used a GST fusion protein encoding its SH3 domain to screen a rat brain cDNA expression library. Of the eighteen positive cDNAs isolated, those encoding synaptojanin 1 were the most abundant. The second most abundant group of cDNAs encoded for the rat germinal center kinase-like kinase (rGLK). It was particularly striking that more independent clones of rGLK were isolated than for the abundant brain protein and established endophilin I-binding partner, dynamin I.

The specificity of the interaction of endophilin I with rGLK was further demonstrated using in vitro binding assays. Whereas the SH3 domain of endophilin I bound strongly to rGLK, it did not bind to the rGLK homologue GCK, and conversely, the proline-rich domain of rGLK bound to endophilin I but not to the abundant SH3 domain-containing proteins, amphiphysin I and II. In fact, overlay assays of brain extracts with the rGLK proline-rich domain detected endophilin I exclusively, suggesting that endophilin I is a major rGLK-binding protein in brain.

Previously, we performed overlays with the SH3 domain of endophilin I that revealed dynamin I and synaptojanin 1 as the major endophilin I-binding partners in brain (Micheva et al., 1997a). However, dynamin I and rGLK co-migrate on SDS-PAGE at 100 kDa so it is possible that the 100 kDa band detected in these experiments represented a mixture of dynamin I and rGLK.

Together, these data suggest a strong and highly specific interaction of endophilin I with rGLK.

The potential significance of the endophilin I/rGLK interaction is highlighted by its occurrence in vivo. Specifically, endophilin I and rGLK can be co-immunoprecipitated following co-transfection in mammalian cells as well as from brain extracts. To further confirm the functional relevance of the interaction, we investigated whether endophilin I expression regulates JNK activation. Overexpression of hGLK in mammalian cells is sufficient to activate JNK (Diener et al., 1997) Interestingly, we find that overexpression of full-length endophilin I increases rGLK-mediated JNK activation in the same system. Further, overexpression of the isolated N-terminus or SH3 domain of endophilin I blocks the activation of JNK stimulated by rGLK.

The mechanism by which endophilin I functions in rGLK-mediated JNK activation is unknown. However, for hGLK, deletion of the regulatory domain, including the SH3 domain-binding sites, results in a kinase that has full catalytic activity but which is significantly impaired in its ability to activate JNK (Diener et al., 1997). This data is consistent with a model in which the regulatory domain targets rGLK for interactions necessary for JNK activation. Thus, it is interesting to speculate that endophilin I may function as an adaptor protein to target rGLK to an upstream activator or to a downstream effector functioning in JNK activation.

In fact, other GCKs appear to undergo specific targeting events. For example, the group 1 GCK family member HPK1 is activated following its recruitment to the EGF receptor via interactions with the SH3 domain-containing adaptor protein Grb2 (Anafi et al., 1997). GCK itself is targeted to the Golgi complex via its interactions with the membrane trafficking protein Rab8 (Ren et al., 1996) Irrespective of the precise mechanism, these data suggest a functional role for endophilin I in GLK activation of JNK.

Previous studies have established a role for endophilin I in clathrin-mediated endocytosis (Simpson et al., 1999; Ringstad et al., 1999; Gad et al., 2000). Our data, demonstrating a functional role for endophilin I in rGLK-mediated JNK activation, suggest an additional, and perhaps complementary role for endophilin I in signaling via the JNK pathway. A link between endocytosis and cell signaling was originally suggested by the observation that active EGF receptor signaling complexes undergo endocytosis and continue to signal on endosomes (Di Guglielmo et al., 1994). It was then demonstrated that endocytosis is necessary for full activation of erk½ MAP kinases following EGF stimulation (Vieira et al., 1996). In fact, recent studies have suggested that a broad range of receptors require endocytosis for erk½ activation including tyrosine-kinase receptors and G-protein-coupled receptors (Ceresa and Schmid, 2000). Endocytosis may be necessary to allow for access of receptors or their downstream signalling components to intracellular pools of erk½ (Kranenburg et al., 1999). Endocytosis may also function in the compartmentalization of signaling complexes, allowing for an additional level of specificity in signaling pathways (DeFea et al., 1999; Zhang et al., 2000). Thus, it is an attractive hypothesis that endophilin I is a component of a pathway providing a novel link between endocytosis and JNK signaling. However, direct evidence to this effect is lacking and the described ability of endophilin I to function in JNK activation may occur independent of its role in endocytosis.

Previous northern blot analysis demonstrated that hGLK has a ubiquitous tissue distribution (Diener et al, 1997). To further characterize the properties of rGLK, we generated a polyclonal antibody against the protein. Extensive analysis of the affinity-purified antibody revealed that it is specific for rGLK. Western blot analysis with this antibody revealed that rGLK protein is expressed in multiple tissues but that its expression is greatly enriched in brain. To determine if rGLK was expressed in neurons, we performed immunofluorescence analysis of rGLK in hippocampal neurons maintained in culture for two days. The neurons displayed strong rGLK staining that was found in puncta that were concentrated in the peri-nuclear region but which extended into neurites including nerve-terminal growth cones. Previously, Ringstad et al (1997) examined the distribution of endophilin I in hippocampal neurons maintained in culture for two weeks and determined that the protein was concentrated in nerve terminals but also displayed a diffuse cytoplasmic staining. Thus, complexes of endophilin I with rGLK may occur in the rGLK-positive vesicular compartment. The identification of the rGLK-positive vesicles is currently under investigation.

The neuronal enrichment of rGLK distinguishes it from most other group I GCKs, which are ubiquitously distributed or are expressed predominantly in lymphoid tissue (Katz et al., 1994; Hu et al., 1996; Kiefer et al., 1996: Diener et al., 1997; Shi and Kehrl, 1997; Fu et al., 1999; Yao et al., 1999; Moore et al., 2000), but is similar to the recently described GCK family member MINK (Dan et al., 2000). Thus, rGLK may play a prominent role in JNK activation in neurons. It was therefore intriguing to observe that in brain, rGLK is expressed in neuronal populations that undergo JNK-mediated neuronal cell death and degeneration (Mielke and Herdegen, 2000). For example, within the hippocampus, the CA3 pyramidal cells strongly express rGLK whereas CA1 pyramidal cells and granule cells of the dentate gyrus show limited or no expression. Endophilin I is also strongly expressed in the cell bodies of CA3 neurons but is detected at much lower levels in the cytoplasm of CA1 neurons (Micheva et al., 1997a). It has long been appreciated that CA3 neurons are prone to excitotoxic cell death (Nadler et al., 1980), a process mediated through activation of the JNK pathway (Yang et al, 1997). Another area with high rGLK expression is the substantia nigra pars compacta that undergoes JNK-mediated apoptotic cell death following axonal damage (Heregen et al, 1998; Oo et al., 1999) or treatment of nigral neurons in vitro with the dopaminergic neuron toxin MPTP (Saporito et al., 1999). Although correlative, these results may suggest a role for rGLK in neuronal cell death.

Another situation in which neurons undergo JNK-mediated apoptotic cell death is in Huntington's disease (HD). HD is caused by an expansion of a CAG repeat (encoding polyglutamine) in the huntingtin protein of HD patients (The Huntington's Disease Collaborative Research Group, 1993). Expression of polyglutamine-expanded huntingtin in a hippocampal cell line induces apoptosis via JNK activation (Liu, 1998; Liu et al., 2000), suggesting a role for JNK activation in the pathophysiology of HD. Adjacent to the polyglutamine region is an SH3 domain-consensus binding site that interacts with endophilin III with greatly increased affinity when huntingtin contains a glutamine expansion in the pathological range (Sittler et al., 1998). Endophilin III is highly related to endophilin I (Sparks et al., 1996; Giachino et al., 1997; Ringstad et al., 1997; So et al., 1997) and in fact, the SH3 domain of endophilin III binds rGLK in vitro (data not shown). Thus, it is intriguing to speculate that in HD, polyglutamine-expanded huntingtin may compete with GLK for binding to endophilin I and/or III leading to a disregulation of JNK signaling via disruption of endophilin/GLK interactions. Studies are underway to determine if there is a direct role for endophilin/GLK interactions in HD.

Drug Screening Assay

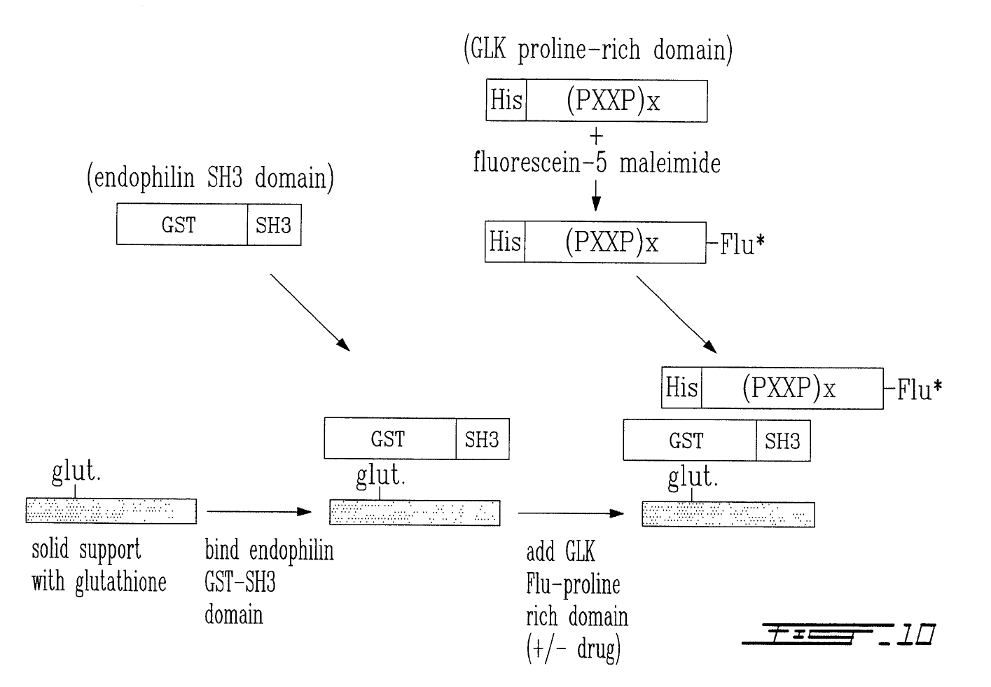

The invention provides methods for detecting agents such as drugs that can alter the ability of members of the endophilin protein family to associate with the germinal center kinase-like kinase (GLK) protein, and methods for detecting agents that induce dissociation of a bound complex formed by the association of an endophilin family member and the GLK protein. An example of a screening assay for detecting such agents is provided in FIG. 10 and is described in the Example 1 below.

As used herein, the term “agent” means a chemical compound that can be useful as a drug. The screening assay described herein is particularly useful in that it can be automated, which allows for high through-put screening of randomly designed agents to identify useful drugs which can alter the ability of the endophilins and GLK to associate. For example, a drug can alter the ability of the endophilin member to associate with GLK by decreasing or inhibiting the binding affinity of the endophilin with GLK. Such a drug could be useful where it is desirable to increase the concentration of unbound GLK in a cell, and therefore modulate the GLK-mediated JNK pathway which may have effects on apoptosis, Alternatively, a drug can be useful for increasing the affinity of binding of endophilin with GLK, that may be desirable for inducing the opposing regulatory effects on the GLK-mediated JNK pathway and apoptosis.

The drug screening assay can utilize an endophilin family member or, as exemplified in FIG. 10, an endophilin fusion protein such as the endophilin SH3 domain glutathione-S-transferase (GST). The endophilin or endophilin fusion protein is characterized, in part, by having an affinity for a solid substrate as well as having an affinity for GLK. For example, when endophilin is used in the assay, the solid substrate can contain a covalently-attached anti-endophilin antibody. Alternatively, if an endophilin-GST fusion protein is used in the assay, the solid substrate can contain covalently-attached glutathione, which is bound by the GST component of the endophilin-GST fusion protein.

The drug screening assay can be performed by allowing the endophilin or endophilin-fusion protein to bind to the solid support, then adding GLK, together with a drug to be tested (see Example 1, below). Control reactions will not contain the drug. Following incubation of the reaction mixture under conditions known to be favorable for the association, for example of the endophilin and GLK in the absence of the drug, the amount of GLK specifically bound to the endophilin in the presence of the drug can be determined. For ease of detection of binding, the GLK protein can be labeled with a detectable moiety, such as a radionuclide or a fluorescent label (see Example 1, below). By comparing the amount of specific binding of the endophilin and GLK in the presence of a drug as compared to the control level of binding, a drug that increases or decreases the binding of the endophilin with GLK can be identified. Thus the drug screening assay provides a rapid and simple method for selecting drugs having a desirable effect on the association of the endophilins with GLK.

### EXAMPLE 1

Screening Assay

This example describes an assay useful for screening for agents such as drugs that alter the affinity of binding of an endophilin family member and the GLK protein. FIG. 10 presents a scheme for using an endophilin family member in a drug screening assay that is suitable for automated high through-put random drug screening. A cDNA encoding the mouse SH3 domain of endophilin I is subcloned into the pGEX-2T prokaryotic expression plasmid (Pharmacia; Piscataway, N.J.) to produce glutathione-S-transferase (GST)endophilin I SH3 domain fusion protein in E. coli. GST-endophilin I SH3 domain fusion protein is affinity purified using glutathione-Sepharose (Sigma Chem. Go.; St. Louis, Mo.). Following loading of the GST-endophilin I SH3 domain, the column is washed with PBS (pH 7.4), containing 1% TX-100, to remove irrelevant proteins. The specific recombinant fusion protein is eluted using excess glutathione in PBS (pH 7.4). Following dialysis, the GST-endophilin I SH3 domain fusion protein is immobilized to solid supports taking advantage of the ability of the GST fusion protein to specifically bind glutathione.

The assay can utilize any form of GLK that includes the proline-rich endophilin-binding site. A cDNA encoding the proline-rich region of GLK is subcloned into the baculovirus transfer vector, pAcSG-His, which produces Histidine-tagged fusion proteins in Sf9 cells (PharMingen, Inc.). The recombinant protein is affinity-purified by standard methods using nickel-chelation chromatography, essentially as described by Smith and Johnson, Gene 67:31-40 (1988), which is incorporated herein by reference. The recombinant GLK fusion protein can be eluted in imidazole (pH 6.0). Following dialysis, the His-GLK fusion protein can be chemically modified to permit easy detection. Several different chemical modifications can be used to attach a detectable moiety such as a fluorescent molecule, a radiolabel or another protein which can be detected using a specific antibody or other specific reagent. For example, fluorescein-5 maleimide can be attached as a fluorescent tag to the GLK protein. Various agents such as drugs are screened for the ability to alter the association of endophilin I and GLK. The agent, endophilin I and fluorescent-GLK are added together, incubated for 30 min to allow binding, then washed to remove unbound fluorescent-GLK protein. The relative amount of binding of fluorescent-GLK protein in the absence as compared to the presence of the agent being screened is determined by detecting the relative light emission of the fluorochrome.

The assay is readily adaptable for examining the interaction of other endophilin family members with GLK, such as endophilin II and III. The screening assay is useful for detecting agents that alter the association of other endophilin family members and the GLK protein by increasing or decreasing their binding affinity.

Glioma Survival is Reduced in the Presence of GLK

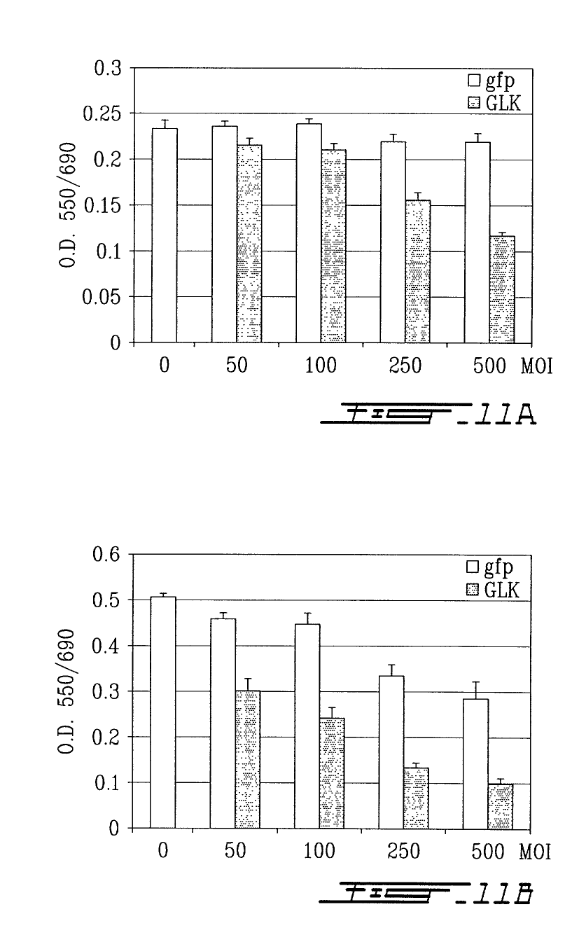

As shown in FIG. 11, glioma cell lines (A) U343 and (B) 373 were plated in 96-well plates, infected with nothing, control GFP adenovirus or GLK adenovirus and analyzed for survival by MTT dye incorporation.

### EXAMPLE 2

Survival Assays

Glioma cell lines U343 and U373 (p53 null) were plated at 5000 cells/well on 96-well plates and infected 24 hours later with increasing MOIs (0, 50, 100, 250 and 500) of recombinant gfp or GLK adenovirus. Appropriate titers of virus were diluted into 10% of the culture volume containing DEAE-dextran, incubated for 30 min at 25° C. and then added directly to the cells. Twenty-four hours post-infection, cells were assayed for viability using 3(4,5-dimethylthiozol-2-yl)2,5-diphenyltetrazolium bromide (MTT; Sigma), which was added at a final concentration of 1 mg/ml for 4 hours. The reaction was ended by the addition of 1 volume of solubilization buffer (20% SDS, 10% dimethylformamide, and 20% acetic acid). After overnight solubilization, specific and non-specific absorbance were read at 550 and 690 nm, respectively, and the average of 6 wells per condition were compared.

Although the present invention has been described hereinabove by way of preferred embodiments thereof, it can be modified, without departing from the spirit and nature of the subject invention as defined in the appended claims.

### REFERENCES

Anafi, M., Kiefer, F., Gish, G. D., Mbamalu, G., Iscove, N. N., and Pawson, T. (1997) J. Biol. Chem. 272, 27804-27811.

Ceresa, B. P., and Schmid, S. L. (2000) Regulation of signal transduction by endocytosis. Curr. Op. Cell Biol. 12, 204-210.

Cestra, G., Castagnoli, L., Dente, L., Minenkova, O., Petrelli, A., Migone, N., Hoffmuller, U., Schneider-Mergener, J., and Cesareni, G. (1999) The SH3 domains of endophilin and amphiphysin bind to the proline-rich region of synaptojanin 1 at sitinct sites that dsiplay an unconventional specificity. J. Biol. Chem. 274, 32001-32007.

Dan, I., Watanabe, N. M., Kobayashi, T., Yamashita-Suzuki, K., Fukagaya, Y., Kajikawa, E., Kimura, W. K., Nakashima, T. M., Matsumoto, K., Ninomiya-Tsuji, J., and Kusumi, A. (2000) Molecular cloning of MINK, a novel member of mammalian GCK family kinases, which is up-regulated during postnatal mouse cerebral development. FEBS Lett. 469, 19-23.

DeFea, K. A., Zalevsky, J., Thoma, M. S., Dery, O., Mullins, R. D., and Bunnett, N. W. (1999) β-Arrestin-dependent endocytosis of proteinase-activated receptor 2 is required for intracellular targeting of activated ERK½. J. Cell Biol. 148, 1267-1281.

de Heuvel, E., Bell, A. W., Ramjaun, A. R., Wong, K., Sossin, W. S., and McPherson, P. S. (1997) Identification of the major synaptojanin-binding proteins in brain. J. Biol. Chem. 272, 8710-8716.

Di Guglielmo, G. M., Baass, P. C., Ou, W-J., Posner, B. I., and Bergeron, J. J. M. (1994) Compartmentalization of SHC, GRB2 and mSOS, and hyperphosphorylation of Raf-1 by EGF but not insulin in liver parenchyma. EMBO J. 13, 4269-4277.

Diener, K., Wang, X. S., Chen, C., Meyer. C. F., Keesler, G., Zukowski, M., Tan, T. H., and Yao, Z. (1997) Activation of the c-Jun N-terminal kinase pathway by a novel protein kinase related to human germinal center kinase. Proc. Natl. Acad. Sci., USA 94, 9687-9692.

Fu, C. A., Shen, M., Huang, B. C., Lasaga, J., Payan, D. G., and Luo, Y. (1999) TNIK, a novel member of the germinal center kinase family that activates the c-Jun N-terminal kinase pathway and regulates the cytoskeleton. J. Biol. Chem. 274, 30729-30737.

Gad, H., Ringstad, N., Low, P., Kjaerulff, O., Gustafsson, J., Wenk, M., Di Paolo, G., Nemoto, Y., Crun, J., Ellisman, M. H., De Camilli, P., Shupliakov, O., and Brodin, L. (2000) Fission and uncoating of synaptic clathrin-coated vesicles are perturbed by disruption of interactions with the SH3 domain of endophilin. Neuron 27, 301-312.

Giachino, C., Lantelme, E., Lanzetti, L., Saccone, S., Bella Valle, G., and Migone, N. (1997) A novel SH3-containing human gene family preferentially expressed in the central nervous system. Genomics. 41, 427-434.

Herdegen, T., Claret. F. X., Kallunki, T., Martin-Villalba, A., Winter, C., Hunter, T., and Karin, M. (1998) Lasting N-terminal phosphorylation of c-Jun and activation of c-Jun N-terminal kinases after neuronal injury. J. Neurosci. 18, 5124-5135.

Howard, L., Nelson, K. K., Maciewicz, R. A., and Blobel, C. P. (1999) Interaction of the metalloprotease disintegrins MDC9 and MDC15 with two SH3 domain-containing proteins, endophilin I and SH3PX1. J. Biol. Chem. 274, 31693-31699.

Hu, M. C., Qiu, W. R., Wang, X., Meyer, C. F., and Tan, T. H. (1996) Human HPK1, a novel human hematopoietic progenitor kinase that activates the JNK/SAPK kinase cascade. Genes Dev. 10, 2251-2264.

Katz, P., Whalen, G., and Kehrl, J. H. (1994) Differential expression of a novel protein kinase in human B lymphocytes. Preferential localization in the germinal center. J. Biol. Chem. 269, 16802-16809.

Kay, B. K., Williamson, M. P., and Sudol, M. (2000) The importance of being proline: the interaction of proline-rich motifs in signaling proteins with their cognate domains. FASEB J. 14, 231-241.

Kiefer, F., Tibbles, L. A., Anafi, M., Janssen, A., Zanle, B. W., Lassam, N., Pawson, T., Woodgett, J. R., and Iscove, N. N. (1996) HPK1, a hematopoietic protein kinase activating the SAPK/JNK pathway. EMBO J. 15, 7013-7025.

Kozak, M. (1991) An analysis of vertebrate mRNA sequences intimations of translational control. J. Cell Biol. 115, 887-903.

Kranenburg, O., Verlaan, I. and Moolenaar, W. H. (1999) Dynamin is required for the activation of mitogen-activated protein (MAP) kinase by MAP kinase kinase. J. Biol. Chem. 274, 35301-35304.

Kyriakis, J. M. (1999) Signaling by the germinal center kinase family of protein kinases. J. Biol. Chem. 274, 5259-5262.

Leprince, C., Romero, F., Cussac, D., Vayssiere, B., Berger, R., Tavitian, A., and Camois, J. H. (1997) A new member of the amphiphysin family connecting endocytosis and signal transduction pathways. J. Biol. Chem. 272, 15101-15105.

Liu, Y. F. (1998) Expression of polyglutamin-expanded Huntingtin activates the SEK1-JNK pathway and induces apoptosis in a hippocampal neuronal cell line. J. Biol. Chem 273, 28873-28877.

Liu, Y. F., Dorow, D., and Marshall, J. (2000) Activation of MLK2-mediated signaling cascades by polyglutamine-expanded huntingtin. J. Biol. Chem. 275, 19035-19040.

McPherson, P. S., Czernik, A. J., Chilcote, T. J., Onofri, F., Benfenati, F., Greengard, P., Schlessinger, J., and De Camilli, P. (1994) Interaction of Grb2 via its Src homology 3 domains with synaptic proteins including synapsin I. Proc. Natl. Acad. Sci., USA. 91, 6486-6490.

McPherson, P. S. (1999) Regulatory role of SH3 domain-mediated protein-protein interactions in synaptic vesicle endocytosis. Cellular Signalling 11, 229-238.

Micheva, K. D., Kay, B. K., and McPherson, P. S. (1997a) Synaptojanin forms two separate complexes in the nerve terminal. Interactions with endophilin and amphiphysin J. Biol. Chem. 272, 27239-27245.

Micheva, K. D., Ramjaun, A. R., Kay, B. K., and McPherson, P. S. (1997b) SH3 domain-dependent interactions of endophilin with amphiphysin. FEBS Lett. 414, 308-312.

Mielke K., and Herdegen, T. (2000) JNK and p38 stresskinases-degenerative effectors of signal-transduction-cascades in the nervous system. Prog. Neurobiol. 61, 45-60.

Moore, T. M., Garg, R., Johnson, C., Coptcoat, M. J., Ridley, A. J., and Morris, J. D. (2000) PSK, a novel STE20-like kinase derived from prostatic carcinoma that activates the c-Jun N-terminal kinase mitogen-activated protein kinase pathway and regulates actin cytoskeletal organization. J. Biol. Chem. 275, 4311-4322.

Nadler, J. V., Perry, B. W., Gentry, C., and Cotman, C. W. (1980) Degeneration of hippocampal CA3 pyramidal cells induced by intraventricular kainic acid. J. Comp. Neurol. 192, 333-359.

Oo, T. F., Henchcliffe, C., James, D., and Burke, R. E. (1999) Expression of c-fos, c-jun, and c-jun N-terminal kinase (JNK) in a developmental model of induced apoptotic death in neurons of the substantia nigra. J. Neurochem. 72, 557-564.

Ramjaun, A. R., Micheva, K. D., Bouchelet, I. B., and McPherson, P. S. (1997) Identification and characterization of a nerve terminal-enriched amphiphysin isoform. J. Biol. Chem. 272, 16700-16706.

Ren, M., Zeng, J., De Lemos-Chiarandini, C., Rosenfeld, M., Adesnik, M., and Sabatini, D. D. (1996) In its active form, the GTP-binding protein rab8 interacts with a stress-activated protein kinase. Proc. Natl. Acad. Sci., USA 93, 5151-5155.

Ringstad, N., Nemoto, Y., and De Camilli, P. (1997) The SH3p4/Sh3p8/SH3p13 protein family; binding partners for synaptojanin and dynamin via a Grb2-like Src homology 3 domain. Proc. Natl. Acad. Sci., USA. 94, 8569-8574.

Ringstad, N., Gad, H., Low, P., Di Paolo, G., Brodin, L., Shupliakov, O., and De Camilli P. (1999) Endophilin/SH3p4 is required for the transition from early to late stages in clathrin-mediated synaptic vesicle endocytosis. Neuron 24, 143-154.

Saporito, M. S., Brown, E. M., Miller, M. S., and Carswell, S. (1999) CEP-1347/KT-7515, an inhibitor of c-jun N-terminal kinase activation, attenuates the 1-methyl-4-phenyl tetrahydropyridine-mediated loss of nigrostriatal dopaminergic neurons In vivo. J. Pharmacol. Exp. Ther. 288, 421-427.

Sharp, A. H., McPherson, P. S., Dawson, T. M, Aoki., C., Campbell, K. P. and Snyder, S. H. (1993) Differential immunohistochemical localizations of inositol 1,4,5-trisphosphate- and ryanodine-sensitive Ca²⁺ release channels in rat brain J. Neurosci. 13, 3051-3063.

Shi, C. S., and Kehrl, J. H. (1997) Activation of stress-activated protein kinase/c-Jun N-terminal kinase, but not NF-kappaB, by the tumor necrosis factor (TNF) receptor 1 through a TNF receptor-associated factor 2- and germinal center kinase related-dependent pathway. J. Biol. Chem. 272, 32102-32107.

Simpson, F., Hussain, N. K., Qualmann, B., Kelly, R. B., Kay, B. K., McPherson, P. S., and Schmid, S. L. (1999) SH3 domain-containing proteins function at distinct steps in clathrin-coated vesicle formation. Nature Cell Biol. 1, 119-124.

Sittler, A., Walter, S., Wedemeyer, N., Hasenbank, R., Scherzinger, E., Eickhoff, H., Bates, G. P., Lehrach, H., and Wanker, E. E. (1998) SH3GL3 associates with the Huntingtin exon 1 protein and promotes the formation of polygln-containing protein aggregates. Mol. Cell 2, 427-436.

Snutch, T. P., Leonard, J. P., Gilbert, M. M., Lester, H. A., and Davidson, N. (1990) Rat brain expresses a heterogeneous family of calcium channels. Proc. Natl. Acad. Sci., USA 87, 3391-339.

So, C. W., Caldas, C., Liu, M. M., Chen, S. J., Huang, Q. H., Gu, L. J., Sham, M. H., Wiedemann, L. M., and Chan, L. C. (1997) EEN encodes for a member of a new family of proteins containing an Src homology 3 domain and is the third gene located on chromosome 19p13 that fuses to MLL in human leukemia. Proc. Natl. Acad. Sci., USA 94, 2563-2568.

Sparks, A. B., Hoffman, N. G., McConnell, S. J., Fowlkes, D. M., and Kay, B. K. (1996) Cloning of ligand targets: systematic isolation of SH3 domain-containing proteins. Nature Biotech. 14, 741-744.

Tang, Y., Hu, L. A., Miller, W. E., Ringstad, N., Hall, R. A., Pitcher, J. A., DeCamilli, P., and Lefkowitz, R. J. (1999) Identification of the endophilins (SH3p4/p8/p13) as novel binding partners for the beta1-adrenergic receptor. Proc. Natl. Acad. Sci., USA 96, 12559-12564.

The Huntington's Disease collaborative Research Group (1993) A Novel gene containing a trinucleotide repeat that is exapnded and unstable on Huntington's disease chromosomes. Cell 72, 971-983.

Tong, X. K., Hussain, N. K., de Heuvel, E., Kurakin, A., Abi-Jaoude, E., Quinn, C. C., Olson, M. F., Marais, R., Baranes, D., Kay, B. K., and McPherson, P. S. (2000) The endocytic protein intersectin is a major binding partner for the Ras exchange factor mSos1 in rat brain. EMBO J. 19, 1263-1271.

Vieira, A. V., Lamaze, C., and Schmid, S. L. (1996) Control of EGF receptor signaling by clathrin-mediated endocytosis. Science 274, 2086-2089.

Yang, D. D., Kuan, C. Y., Whitmarsh, A. J., Rincon, M., Zheng, T. S., Davis, R. J., Rakic, P., and Flavell, R. A. (1997) Absence of excitotoxicity-induced apoptosis in the hippocampus of mice lacking the Jnk3 gene. Nature 389, 865-870.

Yao, Z., Zhou, G., Wang, X. S., Brown, A., Diener, K., Gan, H., and Tan, T. H. (1999) A novel human STE20-related protein kinase, HGK, that specifically activates the c-Jun N-terminal kinase signaling pathway. J. Biol. Chem. 274, 2118-2125.

Yuasa, T., Ohno, S., Kehrl, J. H., and Kyriakis, J. M. (1998) Tumor necrosis factor signaling to stress-activated protein kinase (SAPK)/Jun NH2-terminal kinase (JNK) and p38. Germinal center kinase couples TRAF2 to mitogen-activated protein kinase/ERK kinase kinase 1 and SAPK while receptor interacting protein associates with a mitogen-activated protein kinase kinase kinase upstream of MKK6 and p38. J. Biol. Chem. 273, 22681-22692.

Zhang, Y., Moheban, D. B., Conway, B. R., Bhattacharyya, A., and Segal, R. A. (2000) Cell surface Trk receptors mediate NGF-induced survival while internalized receptors regulate NGF-induced differentiation. J. Neurosci. 20, 5671-5678.

What is claimed is:

1. A method for the identification of an agent that can alter the ability of an endophilin family member or endophilin fusion protein to associate with the germinal center kinase-like kinase (GLK) protein, comprising the steps of;
(a) in a reaction mixture, allowing said endophilin family member or endophilin fusion protein, which is characterized by having an affinity for a solid substrate as well as having an affinity for GLK, to bind to a solid substrate;
(b) adding GLK together with an agent to be tested to the reaction mixture of (a) to form a second reaction mixture;
(c) allowing the second reaction mixture of (b) to incubate; and
(d) measuring the association of said endophilin family member or said endophilin fusion protein with GLK in the presence of said agent to be tested, and comparing same under conditions when said agent to be tested is absent from the second reaction mixture.

2. A method as defined in claim 1, wherein said endophilin family member or endophilin fusion protein is selected from the group consisting of endophilin I, endophilin II, endophilin III and glutathione-S-transferase (GST)-endophilin I SH3 domain fusion protein.

3. A method as defined in claim 1 or 2, wherein (d) is carried out by labeling said GLK protein with a detectable moiety.

4. A method as defined in claim 3, wherein said detectable moiety is a radionuclide, an antibody or fluorescent label.

5. A method as defined in claim 1 or 2, which is automated.

6. Use of a method as defined in claim 5 for high through-put screening of a number of agents.
